(19) United States
(12) Patent Application Publication
(10) Pub. No.: US 2024/0299377 A1
(43) Pub. Date: Sep. 12, 2024

(54) METHODS AND COMPOSITIONS FOR TREATING IRON DISORDERS

(71) Applicant: BeyondSpring Pharmaceuticals, Inc., New York, NY (US)

(72) Inventors: Ramon Mohanlal, New York, NY (US); Lan Huang, New York, NY (US)

(21) Appl. No.: 17/754,894

(22) PCT Filed: Oct. 13, 2020
(86) PCT No.: PCT/US2020/055357

§ 371 Date: Apr. 14, 2022

Related U.S. Application Data
(60) Provisional application No. 62/915,241, filed on Oct. 15, 2019.

(51) Int. Cl. A61K 31/496; A61K 31/132; A61K 31/16; A61K 31/194; A61K 31/4196; A61K 31/44; A61P 3/12

(52) U.S. Cl. CPC A61K 31/496; A61K 31/132; A61K 31/16; A61K 31/194; A61K 31/4196; A61K 31/44; A61P 3/12

(57) ABSTRACT

Disclosed herein are methods of reducing, preventing, or ameliorating tissue iron overload and disorders associated with depletion/reduction of plasm haptoglobin levels in the presence of increased plasma hemoglobin levels due to hemolysis. In particular, some embodiments relate to administering a clinically relevant amount of plinabulin to a subject.

### CROSS REFERENCE TO RELATED APPLICATIONS

This patent application claims the benefit of priority to U.S. Provisional Application No. 62/915,241, filed Oct. 15, 2019. All of the foregoing applications are fully incorporated herein by reference in their entireties for all purposes.

### FIELD

The present disclosure relates to the field of chemistry and medicine. More specifically, it relates to methods of treating, preventing, and ameliorating iron disorders using plinabulin.

### BACKGROUND

Iron is crucial for maintaining normal structure and function of virtually all mammalian cells. Iron is normally stored intracellularly in the form of ferritin, a protein whose synthesis is induced upon influx of iron. When the storage capacity of ferritin is exceeded, pathological quantities of metabolically active iron are released intracellularly in the form of hemosiderin (partially degraded ferritin) and free iron within an expanded labile pool. This metabolically active iron catalyzes the formation of free radicals, which damage membrane lipids and other macromolecules, leading to cell death and eventually organ failure. The heart is more susceptible to iron toxicity than the liver because the liver produces much more ferritin.

A complication of chronic blood transfusions for chronic anemia is the increase of free hemoglobin levels and subsequent iron overload in tissues (such as myocardium, liver, endocrine organs etc.), leading to morbidity and shortened life expectancy of patients as young as 2 year old. A number of conditions such as SCD, MDS, hematological malignancies undergoing stem cell transplantation, chronic renal dysfunction require frequent blood transfusions. Free plasma hemoglobin is bound rapidly and irreversibly by haptoglobin through strong non-covalent bonds. This haptoglobin-hemoglobin complex is then removed by macrophages via a cell-surface receptor (CD163), leading to depletion of plasma haptoglobin levels. Efficient removal of free hemoglobin is essential for health because of the oxidative and toxic properties of the iron-containing heme in hemoglobin (Kristiansen Nature 2001). Iron overload is currently treated by iron chelation therapy, which are only indicated once iron overload in tissues already have reached a critical threshold level. Currently there are no effective treatments available to prevent iron overload.

### SUMMARY OF THE INVENTION

In object of the present disclosure is a method of preventing an iron disorder in a mammal is disclosed. In some embodiments, the method comprises administering to the mammal in need thereof a therapeutically effective amount of plinabulin.

In some embodiments, the iron disorder is associated with chronic blood transfusions. In some embodiments, the iron disorder is associated with disease. In some embodiments, the disease is selected from the group consisting of neurodegenerative diseases, cardiovascular diseases, inflammatory diseases, cancer, insulin resistance, non-alcoholic liver disease, alcoholic liver disease, and an infectious disease. In some embodiments, the neurodegenerative disease is ALS, a prion disease, Parkinson's disease, or Alzheimer's disease. In some embodiments, the cardiovascular disease is atherosclerosis, ishemic cerebrovascular disease, or ischemic stroke. In some embodiments, the cancer is head and neck cancer, lung cancer, stomach cancer, colon cancer, pancreatic cancer, prostate cancer, breast cancer, kidney cancer, bladder cancer, ovary cancer, cervical cancer, melanoma, glioblastoma, myeloma, lymphoma, leukemia, renal cell carcinoma, malignant melanoma, non-small cell lung cancer (NSCLC), ovarian cancer, Hodgkin's lymphoma or squamous cell carcinoma.

In some embodiments, the iron disorder is selected from iron overload disorder or an anemic condition. In some embodiments, the iron overload is associated with oxyradical formation. In some embodiments, the oxyradical formation is associated with an ischemia-reperfusion. In some embodiments, the ischemia-reperfusion occurs during an organ transplant, cardiac PCI procedure, and reperfusion procedures.

In some embodiments, the iron overload disorder is a primary iron overload disorder. In some embodiments, the primary iron overload disorder is selected from the group consisting of hereditary hemochromatosis, juvenile hemochromatosis, ferroportin disease, neonatal hemochromatosis, Bantu siderosis, African iron overload, gracile syndrome, ataxia, and Friedreich Ataxia. In some embodiments, the iron overload disorder is a secondary iron overload disorder. In some embodiments, the secondary iron overload disorder is selected from thalassemia, hypochromic microcytic anemia, sickle cell anemia, microcytic iron loading anemia, hereditary sideroblastic anemia, congenital dyserythropocitic anemia, Porphyria cutanea tarda, pyruvate kinase deficiency, hereditary atransferrinemia, ceruloplasmin deficiency, myelodysplastic syndromes, pulmonary hemosiderosis, aceruloplasminemia and x-linked sideroblastic anemia. In some embodiments, the anemic condition is selected from the group consisting of hemolytic anemia, thalassemia, hereditary spherocytosis, hereditary elliptocytosis, glucose-6-phosphate dehydrogenase deficiency, pyruvate kinase deficiency, immune hemolytic anemia, alloimmune hemolytic anemia, drug-induced hemolytic anemia, mechanical hemolytic anemia, paroxysmal nocturnal hemoglobinuria, anemia of chronic disease, anemia, aplastic anemia, congenital hypoplastic anemia, Diamond-Blackfan anemia, Fanconi anemia, iron deficiency anemia, anemia of abnormal RBC size, megaloblastic anemia, microcytic anemia, vitamin deficiency anemia, pernicious anemia, anemia of RBC mutation, sideroblastic anemia, and sickle cell anemia.

In some embodiments, the iron overload is an accumulation of iron in a body tissue. In some embodiments, the body tissue is selected from myocardium, liver, and endocrine organs. In some embodiments, the method further administers a metal chelator. In some embodiments, the metal chelator is an iron chelator. In some embodiments, the iron chelator is selected from is deferasirox, dimercaptosuccinic acid, deferoxamine, deferiprone, or trientine. In some embodiments, the mammal exhibits an elevated haptoglobin level. In some embodiments, prior to treatment, the mammal exhibits a decreased hemoglobin level. In some embodiments, the method further identifies the mammal as having the iron disorder prior to administration of plinabulin.

In some aspects, a method of increasing haptoglobin in a mammal is disclosed. The method comprises administering to the mammal in need thereof a therapeutically effective amount of plinabulin. In some embodiments, the method identifies the mammal as having a decreased haptoglobin level prior to administration of plinabulin.

In some aspects, a method of reducing iron overload is disclosed. The method comprises administering plinabulin or a pharmaceutically acceptable salt thereof to a subject in need thereof. In some embodiments, the method further identifies the subject as having iron overload prior to administration of plinabulin.

In some aspects, a method of decreasing free plasma hemoglobin in a subject is disclosed. The method comprises administering plinabulin or a pharmaceutically acceptable salt thereof to a subject in need thereof. In some embodiments, the method identifies the subject as having an elevated free plasma hemoglobin level prior to administration of plinabulin.

In some aspects, a method of treating an iron disorder is disclosed. The method comprises administering plinabulin or a pharmaceutically acceptable salt thereof to a subject in need thereof. In some embodiments, the method further comprises identifying the subject as at risk for developing the iron disorder prior to administration of plinabulin.

In some embodiments, the administration of plinabulin reduces an incidence of the iron disorder by at least 10%. In some embodiments, the administration of plinabulin reduces an incidence of the iron disorder by at least 30%. In some embodiments, the administration of plinabulin reduces a duration of the iron disorder by at least 2 times. In some embodiments, the administration of plinabulin reduces a duration of the iron disorder by at least 30%. In some embodiments, the plinabulin is administered at a dose in the range of about 2.5 mg/m² to about 40 mg/m². In some embodiments, the plinabulin is administered at a dose in the range of about 8.5 mg/m² to about 35 mg/m². In some embodiments, the plinabulin is administered at a dose in the range of about 20 mg/m² to about 35 mg/m². In some embodiments, the plinabulin is administered at a dose in the range of about 25 mg/m² to about 35 mg/m². In some embodiments, the plinabulin is administered at a dose about 30 mg/m². In some embodiments, the plinabulin is administered simultaneously with a blood transfusion. In some embodiments, the plinabulin is administered prior to a blood transfusion. In some embodiments, the plinabulin is administered after a blood transfusion.

### BRIEF DESCRIPTION OF THE DRAWINGS

FIG. 1 illustrates a Plinabulin treatment over 22 days and haptoglobin change from baseline results.

### DETAILED DESCRIPTION

Disclosed herein are compositions and methods useful for the prevention, treatment, and/or amelioration of various diseases, disorders, or conditions. Some embodiments pertain to a composition comprising plinabulin. Plinabulin, (3Z,6Z)-3-Benzylidene-6-{[5-(2-methyl-2-propanyl)-1H-imidazol-4-yl]methylene}-2,5-piperazinedione, is a synthetic analog of the natural compound phenylahistin. Plinabulin can be readily prepared according to methods and procedures detailed in U.S. Pat. Nos. 7,064,201 and 7,919,497, which are incorporated herein by reference in their entireties. Some embodiments relate to the use of plinabulin or a pharmaceutically effective salt thereof to treat, prevent, or ameliorate an iron disorder. Also presented herein is the surprising discovery that exposing a subject to a composition as disclosed herein can increase haptoglobin levels.

The section headings used herein are for organizational purposes only and are not to be construed as limiting the subject matter described. Features disclosed under one heading (such as a composition) can be used in combination with features disclosed under a different heading (a method of treating). Unless defined otherwise, all technical and scientific terms used herein have the same meaning as is commonly understood by one of ordinary skill in the art. It should be noted that the use of particular terminology when describing certain features or aspects of the disclosure should not be taken to imply that the terminology is being re-defined herein to be restricted to include any specific characteristics of the features or aspects of the disclosure with which that terminology is associated.

While the disclosure has been illustrated and described in detail in the foregoing description, such description is to be considered illustrative or exemplary and not restrictive. The disclosure is not limited to the disclosed embodiments. Variations to the disclosed embodiments can be understood and effected by those skilled in the art in practicing the claimed disclosure, from a study of the disclosure and the appended claims.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

As used in the specification and the appended claims, the singular forms “a,” “an” and “the” include plural referents unless the context clearly dictates otherwise. The use of “or” or “and” means “and/or” unless stated otherwise. Furthermore, use of the term “including” as well as other forms, such as “include”, “includes,” and “included,” is not limiting. The mere fact that certain measures are recited in mutually different dependent claims does not indicate that a combination of these measures cannot be used to advantage.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value. Where particular values are described in the application and claims, unless otherwise stated the term “about” meaning within an acceptable error range for the particular value should be assumed.

As used herein, a “carrier” as used herein is a broad term, and is to be given its ordinary and customary meaning to a person of ordinary skill in the art (and is not to be limited to a special or customized meaning), and refers without limitation to a compound that facilitates the incorporation of a compound into cells or tissues. For example, without limitation, dimethyl sulfoxide (DMSO) is a commonly utilized carrier that facilitates the uptake of many organic compounds into cells or tissues of a subject. Water, saline solution, ethanol, and mineral oil are also carriers employed in certain pharmaceutical compositions.

As used herein, a “diluent” as used herein is a broad term, and is to be given its ordinary and customary meaning to a person of ordinary skill in the art (and is not to be limited to a special or customized meaning), and refers without limitation to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

As used herein, an “excipient” as used herein is a broad term, and is to be given its ordinary and customary meaning to a person of ordinary skill in the art (and is not to be limited to a special or customized meaning), and refers without limitation to a substance that is added to a pharmaceutical composition to provide, without limitation, bulk, consistency, stability, binding ability, lubrication, disintegrating ability etc., to the composition. A “diluent” is a type of excipient.

As used herein. “iron disorder” refers to a condition in a mammal, preferably a human, wherein the level of iron in the body is outside the normal range for the particular mammal (i.e. abnormal iron level), such as an elevated or a decreased iron serum level compared to the normal iron serum level for the mammal or an increased or decreased level of iron in the liver of the mammal as compared to the normal level of iron in the liver in the mammal.

As used herein, “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

As used herein, “pharmaceutically acceptable” is a broad term, and is to be given its ordinary and customary meaning to a person of ordinary skill in the art (and is not to be limited to a special or customized meaning), and refers without limitation to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable risk/benefit ratio.

The terms “pharmaceutically acceptable salts” and “a pharmaceutically acceptable salt thereof” as used herein are broad terms, and are to be given their ordinary and customary meaning to a person of ordinary skill in the art (and is not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl derivatives, and protected derivatives can also be suitable for use in compositions and methods of preferred embodiments. While it may be possible to administer the compounds of the preferred embodiments in the form of pharmaceutically acceptable salts, it is generally preferred to administer the compounds in neutral form.

The term “pharmaceutical composition” as used herein is a broad term, and is to be given its ordinary and customary meaning to a person of ordinary skill in the art (and is not to be limited to a special or customized meaning), and refers without limitation to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids or bases. Pharmaceutical compositions will generally be tailored to the specific intended route of administration.

As used herein, a “subject” as used herein is a broad term, and is to be given its ordinary and customary meaning to a person of ordinary skill in the art (and is not to be limited to a special or customized meaning), and refers without limitation to an animal that is the object of treatment, observation or experiment. “Animal” includes cold- and warm-blooded vertebrates and invertebrates such as fish, shellfish, reptiles and, in particular, mammals. “Mammal” includes, without limitation, dolphins, mice, rats, rabbits, guinea pigs, dogs, cats, sheep, goats, cows, horses, primates, such as monkeys, chimpanzees, and apes, and, in particular, humans. In some embodiments, the subject is human.

As used herein, “therapeutically effective amount” and “effective amount” as used herein are broad terms, and are to be given its ordinary and customary meaning to a person of ordinary skill in the art (and are not to be limited to a special or customized meaning), and are used without limitation to indicate an amount of an active compound, or pharmaceutical agent, that elicits the biological or medicinal response indicated. For example, a therapeutically effective amount of compound can be the amount needed to prevent, alleviate or ameliorate markers or symptoms of a condition or prolong the survival of the subject being treated. This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

As used herein, “Treating” or “treatment”, covers the treatment of an iron disorder in a mammal, preferably a human, or a disease or condition associated with an iron disorder in a mammal, preferably a human, and includes: (i) preventing an iron disorder in a mammal, or a disease or condition associated with an iron disorder in the mammal, from occurring in the mammal; (ii) inhibiting an iron disorder in a mammal, or a disease or condition associated with an iron disorder in the mammal, i.e., arresting its development; (iii) relieving an iron disorder in a mammal, or a disease or condition associated with an iron disorder in the mammal, i.e., causing regression of the iron disorder or the disease or condition; (iv) relieving the symptoms of an iron disorder in a mammal, or a disease or condition associated with an iron disorder in the mammal, i.e., relieving the symptoms without addressing the underlying iron disorder, disease or condition; or (v) restoring and/or maintaining normal serum iron levels, transferrin saturation, serum ferritin, liver iron and/or bodily iron levels in a mammal having an iron disorder or having a disease or condition associated with an iron disorder.

The term “increase” referred to in the disclosed embodiments refers to an “expected increase” for the subject as opposed to the actual increase any particular subject experiences. Thus, one does not need to wait for a subject's longevity, survival time, life span, or health span to expire in order to practice the disclosed embodiments. Expected increases may be statistically significant or insignificant, though in some embodiments any expected increase is statistically significant. There are many methods known for calculating statistical significance, e.g., calculating a “p-value.” In some embodiments, the threshold for statistical significance is a p-value≤0.2, ≤0.15, ≤0.1, ≤0.05, ≤0.01, ≤0.005, about ≤0.2, about ≤0.15, about ≤0.1, about ≤0.05, about ≤0.01, or about ≤0.005. Sometimes, a result may not be statistically significant but yet the result is still informative or suggestive of some conferred benefit. It is understood that the degree of significance one would ascribe to a particular result is within the ken of the ordinarily skilled physician.

Any percentages, ratios or other quantities referred to herein are on a weight basis, unless otherwise indicated.

### Method of Treatment

Methods of treating, preventing, or ameliorating an iron disorder in a subject are disclosed herein. A subject in need of receiving a composition as disclosed herein to treat, prevent, or ameliorate an iron disorder in a subject need not always be identified prior to receiving a first treatment with the composition. For example, a subject may be predetermined that they will develop an iron disorder prior to showing any signs of an iron disorder. Alternatively, the subject may receive treatment prophylactically if he or she is at risk or not at risk of an iron disorder. Accordingly, in some embodiments, the composition is administered to the subject after the subject receives an early stage diagnosis. In some embodiments, not every subject is a candidate for such administration and identification of treatment in a subject may be desirable. It is understood that patient selection depends upon a number of factors within the skill of the ordinarily skilled physician. Thus, some embodiments, disclosed herein further comprise identifying a subject as one that will benefit from administering an effective amount of at least one compound or composition to treat, prevent or ameliorate an iron disorder. Subjects may be identified on the basis of physiological factors specific to the subject according to the subject's age, present medical condition, present medical treatment, prescribed medical treatment, or in some embodiments, the subject being diagnosed with an iron disorder.

Some embodiments of treating, preventing, or ameliorating an iron disorder include administering to the subject an effective amount of plinabulin. In other embodiments, the disease or condition associate with the iron disorder is due to an accumulation of iron in the body tissues of the mammal. In some embodiments, the iron disorder includes iron deficiency disorders. In some embodiments, the iron disorder includes an iron overload disorders. In some embodiments, iron disorder include two or more conditions associated with an iron disorder. In some embodiments, the method includes administering a therapeutically effective amount of plinabulin and one or more additional therapeutic agents. In some embodiments, the method comprises testing a subject for an iron disorder risk factor and administering a composition as described herein. In some embodiments, testing a subject for an iron disorder risk factor include testing a subject's blood. In some embodiments, testing a subject for an iron disorder risk factor include liver test, MRI scans, and genetic testing. In some embodiments, testing a subject for an iron disorder risk factor include performing a serum ferritin test.

In another aspect, the disclosure provides methods for preventing a disease or condition associated with an iron disorder in a mammal from the accumulation of iron in the body tissues of the mammal, wherein the methods comprise administering to the mammal in need thereof a therapeutically effective amount of plinabulin. In some embodiments, the method includes administering a therapeutically effective amount of plinabulin and one or more additional therapeutic agents. One can readily and immediately envision a regimen wherein a subject is administered a first composition, and the subject receives one or more subsequent composition dosages. Such a regimen may continue such that the subject receives a third composition dosage after the subject receives the second composition dosage. In some embodiments, a subject may receive: 1, 2, 3, 4, 5, 6, 7, 8, 9, 10 or more doses during treatment. In some embodiments, a subject receives a therapeutically effective amount of plinabulin before an iron disorder is achieved.

In another aspect, the iron disorder is an iron overload disorder. In some embodiments, iron overload may also be responsible for a portion of the pathology observed in neurodegenerative diseases (including ALS, prion diseases, Parkinson's, Alzheimer's disease), cardiovascular diseases (including atherosclerosis, ischemic cerebrovascular disease and ischemic stroke), inflammatory diseases and conditions (including arthritis and disease progression in viral hepatitis), cancer, insulin resistance, non-alcoholic liver disease, alcoholic liver disease, and infectious disease (including HIV, malaria and Yersinia infections). In some embodiments, the iron overload is associated with oxyradical formation. In some embodiments, the oxyradical formation is associated with an ischemia-reperfusion. In some embodiments, the ischemia-reperfusion occurs during an organ transplant, cardiac PCI procedure, or reperfusion procedures.

In some aspects, the iron disorder is a primary iron overload disorder. In some embodiments, the iron overload disorder is independently selected from the group consisting of hereditary hemochromatosis, juvenile hemochromatosis, ferroportin disease, neonatal hemochromatosis, Bantu siderosis, African iron overload, gracile syndrome, ataxia, and Friedreich Ataxia as well as all of the anemias listed below in which patients may not be transfused but may become iron overloaded due to increased erythroid drive and the resulting increased iron absorption in the gut) and secondary (or transfusional) iron overload disorder which can be caused by repeated transfusions used to treat a number of distinct anemias, including, but not limited to, thalassemia (beta and alpha, major, minor and intermedia), hypochromic microcytic anemias, sickle cell anemia, microcytic iron loading anemias, hereditary sideroblastic anemias, congenital dyserythropocitic anemias, Porphyria cutanea tarda, pyruvate kinase deficiency, hereditary atransferrinemia, ceruloplasmin deficiency, myelodysplastic syndromes, pulmonary hemosiderosis, aceruloplasminemia and x-linked sideroblastic anemia.

In some aspects, the iron disorder is a secondary iron overload disorder. Another embodiment of this aspect is where the disease or condition is independently selected from the group consisting of thalassemia (beta and alpha, major, minor and intermedia), hypochromic microcytic anemia, sickle cell anemia, microcytic iron loading anemia, hereditary sideroblastic anemia, congenital dyserythropocitic anemia, Porphyria cutanea tarda, pyruvate kinase deficiency, hereditary atransferrinemia, ceruloplasmin deficiency, myelodysplastic syndromes, pulmonary hemosiderosis, aceruloplasminemia and x-linked sideroblastic anemia.

In some embodiments, the mammal includes a subject undergoing bone marrow stem cell translation, for example, requiring multiple blood transfusions. In some embodiments, the mammal includes a subject having sickle cell disease, thalassemia, MDS, or renal dysfunction. In some embodiments, such patients require frequent blood transfusions.

In some aspects, disclosed herein relate to a method of preventing a condition associated with anemic condition. In some embodiments, the method includes administering a therapeutically effective amount of plinabulin. In some embodiments, the anemic condition is associated with anemia (including but not limited to thalassemias, hereditary spherocytosis, hereditary elliptocytosis, glucose-6-phosphate dehydrogenase deficiency, pyruvate kinase deficiency, immune hemolytic anemia, alloimmune hemolytic anemia, drug-induced hemolytic anemia, mechanical hemolytic anemias, and paroxysmal nocturnal hemoglobinuria), anemia of chronic disease, anemia, aplastic anemias (including but not limited to congenital hypoplastic anemia, Diamond-Blackfan anemia and Fanconi anemia), iron deficiency anemia, anemias of abnormal RBC size (including but not limited to megaloblastic anemia and microcytic anemia), vitamin deficiency anemias (including but not limited to pernicious anemia) anemia of RBC mutation (including but not limited to thalassemia, sideroblastic anemia and sickle cell anemia).

In some embodiments, the compositions and methods provided herein are indicated for treatment, prevention and or maintenance of conditions, disorders, diseases and defects associated with iron disorders. In some embodiments, the iron disorder include caused by anemia. In some embodiments, the compositions and methods provided herein are indicated for treatment, prevention and or maintenance of conditions, disorders, diseases and defects associated with iron disorder. In some embodiments, the iron disorders include anemia of chronic disease. In some embodiments, the compositions and methods provided herein are indicated for treatment, prevention and or maintenance of conditions, disorders, diseases and defects associated with iron disorders caused by anemia.

Risk factors of anemia include, but are not limited to, a diet lacking in folate and soluble iron menstruation, pregnancy, chronic conditions, genetic conditions, family history and age. Indicators of anemia, individually or in any combination, which may but need not present, include reduced oxygen transport in the blood, fatigue and diminished physical capacity, or secondary organ dysfunction or damage, including heart arrhythmias and heart failure. In some instances, anemia can be identified by small or pale RBCs. Biomarker indices of anemia include indices and markers provided herein, and other indications available to clinicians, and include, for example, red blood cell count, reticulocytes, hematocrit or packed cell volume, red blood cell distribution width, mean corpuscular hemoglobin concentration (MCHC), mean corpuscular volume (MCV), mean corpuscular hemoglobin (MCH), hemoglobin, white cell count, iron deficiency, serum iron, total iron-binding capacity, transferrin saturation, zinc protoporphyrin, and serum sTfRs.

Aplastic anemia is associated with low RBC production. Iron deficiency anemia is associated with a deficiency in iron stores such that erythropoiesis is inadequate. Sickle cell anemia may be caused by a mutation which results in mutant sickle hemoglobin. Sideroblastic anemias are a group of disorders with the common features of mitochondrial iron accumulation in bone marrow, ineffective erythropoiesis, and increased tissue iron levels. Fanconi anemia refers to a rare genetic disorder associated with a high frequency of hematological abnormalities and congenital anomalies. Vitamin deficiency anemia refers to a lack of healthy red blood cells caused by lower than normal amounts of certain vitamins. Vitamins linked to vitamin deficiency anemia include folate, vitamin B-1, or Vitamin C. Microcytic anemias are characterized by the production of red blood cells that are smaller than normal. It is believed that microcytic anemia can be caused by one or more of a lack of globin product (thalassemia), restricted iron delivery to a heme group of hemoglobin, and defects in the synthesis of the heme group.

In some embodiments, anemic subjects appropriate for treating using the composition and methods provided herein may be identified by the risk factors, indices and markers provided herein, and by other indications available to clinicians, and are characterized by, e.g., low red blood cell count, reticulocytes, hematocrit/packed cell volume, red blood cell distribution width, and hemoglobin; high white blood cell count, iron deficiency, serum iron, total iron-binding capacity, transferrin saturation, zinc protoporphyrin, and serum sTfRs. In some instances, anemia can be identified by small or pale RBCs.

In some aspects, a method of treating a symptom or condition of an iron disorder includes administering an effective amount of plinabulin. In some embodiments, the method reduces a symptom or condition of an iron disorder includes administering an effective amount of plinabulin. In some embodiments, the symptom or condition may include diabetes, elevated liver enzymes, erectile dysfunction, extreme tiredness, heart disease, joint disease, or a combination thereof. In some embodiments, administering an effective amount of plinabulin reduces the symptom or condition by at least 1%, 5%, 10%. 15%, 20%, 25%, 30%, 35%, or ranges including and/or spanning the aforementioned values.

In some aspects, a method of treating, reducing, or ameliorating an iron disorder in a subject receiving a cancer drug includes administering an effective amount of plinabulin. In some embodiments, the method of treating, reducing, or ameliorating an iron disorder in a subject receiving a taxane includes administering a therapeutically effective amount of plinabulin. In some embodiments, the taxane is selected from paclitaxel, docetaxel, cabazitaxel, or a combination thereof. In some embodiments, the method of treating, reducing, or ameliorating an iron disorder in a subject receiving TC (docetaxel and cyclophosphamide) includes administering a therapeutically effective amount of plinabulin. In some embodiments, the method of treating, reducing, or ameliorating an iron disorder in a subject receiving TAC (taxotere, doxorubicin, cyclophosphamide) includes administering a therapeutically effective amount of plinabulin. In some embodiments, the therapeutic amount of plinabulin administered is at a dose from about 10 mg/m² to about 50 mg/m². In some embodiments, the plinabulin is administered on day 1 of a 21 day chemotherapy cycle. In some embodiments, plinabulin is administered only once per cycle. In some embodiments, the subject haptoglobin levels are increased after administration of plinabulin. In some embodiments, the haptoglobin levels are increased by at least 2-fold. In some embodiments, the haptoglobulin levels increase within two days of administering plinabulin. In some embodiments, the increased haptoglobulin levels last for at least 1 week, at least 2 weeks, at least 3 weeks, at least 4 weeks, or ranges including and/or spanning the aforementioned values. In some embodiments, the increase in haptoglobin neutralizes free hemoglobin in a subject. In some embodiments, the increase in haptoglobin levels in a subject prevent, reduce, or ameliorate hemolysis dependent overload.

In some embodiments, the compounds and methods provided herein may provide a decrease below a baseline value (e.g., pretreatment value in a patient being treated, or general value observed in a particular patient population) in a serum or plasma concentration of iron. For example, a serum iron concentration may be decreased by at least about 0.01 μg/dl, at least about 0.05 μg/dl, at least about 0.1 μg/dl, at least about 0.4 μg/dl. 1 μg/dl, at least about 2 μg/dl, at least about 3 μg/dl, at least about 4 μg/dl, at least about 5 μg/dl, at least about 6 μg/dl, at least about 7 μg/dl, at least about 8 μg/dl, at least about 9 μg/dl, at least about 10 μg/dl, at least about 15 μg/dl, at least about 20 g/dl, at least about 25 μg/dl, at least about 30 μg/dl, at least about 35 μg/dl, at least about 40 μg/dl, at least about 45 μg/dl, at least about 50 μg/dl, or more than 50 μg/dl, or ranges including and/or spanning the aforementioned values. In some embodiments, a serum iron concentration may decrease below a baseline value (for example, pretreatment value in a patient being treated, or general value observed in a particular patient population) by at least about 0.01%, at least about 0.05%, at least about 0.1%, at least about 0.5%, at least about 1%, at least about 5%, at least about 10%, at least about 15%, at least about 20%, at least about 25%, at least about 30%, at least about 40%, at least about 50%, at least about 60%, at least about 70%, at least about 80%, or ranges including and/or spanning the aforementioned values.

In some embodiments, the use of plinabulin can reduce the incidence of an iron disorder by at least about 1%, 5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 95%, 100%, or ranges including and/or spanning the aforementioned values. In some embodiments, the use of plinabulin can reduce the incidence of an iron overload by at least about 1%, 5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 95%, 100%, or ranges including and/or spanning the aforementioned values. In some embodiments, the use of plinabulin can reduce the incidence of a primary iron overload by less than about 1%, 5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 95%, 100%, or ranges including and/or spanning the aforementioned values. In some embodiments, the use of plinabulin can reduce the incidence of a secondary iron overload by less than about 1%, 5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 95%, 100%, or ranges including and/or spanning the aforementioned values.

In some embodiments, a method as provided herein may provide a decrease in plasma hemoglobin level. In some embodiments, a method provided herein may provide a decrease in free hemoglobin. For example, the hemoglobin level may decrease below a baseline value (e.g., pretreatment value in a patient being treated, or general value observed in a particular patient population) by at least about 0.1 g/dL, at least about 0.2 g/dL, at least about 0.5 g/dL, at least about 1 g/dL, at least about 5 g/dL, at least about 10 g/dL, at least about 25 g/dL, or at least about 30 g/dL, or at least about 50 g/dL, or at least about 100 mg/dL, or at least about 200 mg/dL, or ranges including and/or spanning the aforementioned values.

In some embodiments, a method provided herein may provide an increase in plasma haptoglobin. In some embodiments, a composition or method provided herein may provide an increase in free haptoglobin. For example, the haptoglobin level may increase above a baseline value (e.g., pretreatment value in a patient being treated, or general value observed in a particular patient population) by at least about 0.1 mg/dL, at least about 0.2 mg/dL, at least about 0.5 mg/dL, at least about 1 mg/dL, at least about 10 mg/dL, at least about 20 mg/dL, at least about 50 mg/dL, or at least about 75 mg/dL, or at least about 100 mg/dL, or at least about 200 mg/dL, or ranges including and/or spanning the aforementioned values. In some embodiments, the use of plinabulin can increase haptoglobin by less than about 1%, 5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 95%, 100%, or ranges including and/or spanning the aforementioned values.

In some embodiments, a method provided herein may provide a decrease in plasma hemopexin. In some embodiments, a method provided herein may provide a decrease in free hemopexin. For example, the hemopexin level may decrease below a baseline value (e.g., pretreatment value in a patient being treated, or general value observed in a particular patient population) by at least about 0.1 mg/dL, at least about 0.2 mg/dL, at least about 0.5 mg/dL, at least about 1 mg/dL, at least about 10 mg/dL, at least about 20 mg/dL, at least about 50 mg/dL, or at least about 75 mg/dL, or at least about 100 mg/dL, or at least about 200 mg/dL, or ranges including and/or spanning the aforementioned values. In some embodiments, the use of plinabulin can reduce hemopexin by less than about 1%, 5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 95%, 100%, or ranges including and/or spanning the aforementioned values.

In some embodiments, the compositions and methods as provided herein modulate a marker of an iron disorder. In certain embodiments, the marker is serum, plasma, or red blood cell membrane iron concentration; serum, plasma, or red blood cell hemoglobin concentration; serum ferritin, serum iron, transferrin saturation, red cell count, reticulocytes, hematocrit or packed cell volume, red blood cell distribution width, mean corpuscular hemoglobin concentration (MCHC), mean corpuscular volume (MCV), mean corpuscular hemoglobin (MCH), hemopexin, heptaglobin white cell count, iron deficiency, serum iron, total iron-binding capacity, transferrin saturation, zinc protoporphyrin, or serum sTfRs.

In some embodiments, the methods provided herein include the step of measuring the concentration of a marker an iron disorder. One of skill in the art will be able to perform suitable methods for such measurements, including but not limited to those described herein. Abnormal iron serum levels can be determined by direct measurement of serum iron using a colorimetric assay, or by the standard transferrin saturation assay (which reveals how much iron is bound to the protein that carries iron in the blood), or by the standard serum ferritin assay. For example, transferrin saturation levels of 45% or higher are usually indicative of abnormally high levels of iron in the serum. Abnormal iron levels in the liver can be determined measuring the iron content of the liver from tissue obtained by a liver biopsy or by imaging technique such as MRI and/or SQUID. The degree of iron levels in other tissues (e.g., brain, heart) may also be estimated using these and other imaging techniques. In some embodiments, an abnormal iron level is an elevated iron level in serum or tissue.

Some embodiments include the use of plinabulin to permit the transfusion of red blood cells that have been stored for prolonged periods of time. For example, various embodiments include transfusing red blood cells that have been stored for longer than 20 days, 25 days, 30 days, 35 days, 40 days, 45 days, or 50 days. Plinabulin may be administered before, after or during transfusion of such red blood cells.

### Dosing

As will be readily apparent to one skilled in the art, the useful in vivo dosage to be administered and the particular mode of administration will vary depending upon the age, weight, the severity of the condition, and mammalian species treated, the particular forms of the compounds employed, and the specific use for which these compounds are employed. The determination of effective dosage levels, that is the dosage levels necessary to achieve the desired result, can be accomplished by one skilled in the art using routine methods, for example, in vivo studies. Reference may be made to, for example, “Estimating the Maximum Safe Starting Dose in Initial Clinical Trials for Therapeutics in Adult Healthy Volunteers,” U.S. Food and Drug Administration, July 2005.

In some embodiments, a method provided herein may comprise administering a therapeutically effective amount of a composition provided herein. In some embodiments, a therapeutically effective amount may be determined by reference to the modulation of a marker of a condition provided herein including an anemic condition. In some embodiments, a therapeutically effective amount may be determined by reference to the modulation of a symptom of a condition provided herein. In still other embodiments, reference may be made to established guidelines for the conditions described herein, including, but not limited to, guidelines for the treatment of a condition provided herein including an anemic condition.

The dosage may vary broadly, depending upon the desired effects and the therapeutic indication, such as marker values. Alternatively, dosages may be based and calculated upon the surface area or weight of the patient, as understood by those of skill in the art. The exact dosage will be determined on a case-by-case basis, or, in some cases, will be left to the informed discretion of the subject.

The dosage may be a single one or a series of two or more given in the course of one or more days, as is appropriate for the individual subject. In some embodiments, the compounds will be administered for a period of continuous therapy, for example for about a week or more (e.g., one week, two weeks, three weeks, four weeks, five weeks, six weeks, seven weeks, eight weeks, or more), for several weeks, for about a month or more (e.g., one month, two months, three months, four months, five months, six months, seven months, eight months, nine months, ten months, eleven months, twelve months, or more), for about a year or more, or for a plurality of years.

As will be understood by those of skill in the art, in certain situations it may be necessary to administer the compounds disclosed herein in amounts that exceed the above-stated, preferred dosage range in order to effectively treat a subject.

Unit dosage forms can also be provided, e.g., individual packages with a premeasured amount of the composition, configured for administration on a predetermined schedule. Unit dosage forms configured for administration one to three times a day are preferred; however, in certain embodiments it may be desirable to configure the unit dosage form for administration more than three times a day, or less than one time per day.

Dosage amount and interval may be adjusted to the individual subject to provide plasma levels of the active moiety which are sufficient to maintain predetermined parameters, indicators, or marker values, or minimal effective concentration (MEC). Dosages necessary to achieve the desired result will depend on individual characteristics and route of administration. However, assays, for example, HPLC assays or bioassays, may be used to determine serum concentrations.

The methods described herein may comprise administering one or more compositions to the selected subject. In some embodiments, the composition is plinabulin or a salt thereof. In some embodiments, plinabulin is administered at a dose in the range of about 1-50 mg/m² of the body surface area. In some embodiments, plinabulin is administered at a dose in the range of about 5 to about 50 mg/m² of the body surface area. In some embodiments, plinabulin is administered at a dose in the range of about 20 to about 40 mg/m² of the body surface area. In some embodiments, plinabulin is administered at a dose in the range of about 15 to about 30 mg/m² of the body surface area. In some embodiments, plinabulin is administered at a dose in the range of about 0.5-1, 0.5-2, 0.5-3, 0.5-4, 0.5-5, 0.5-6, 0.5-7, 0.5-8, 0.5-9, 0.5-10, 0.5-11, 0.5-12, 0.5-13, 0.5-13.75, 0.5-14, 0.5-15, 0.5-16, 0.5-17, 0.5-18, 0.5-19, 0.5-20, 0.5-22.5, 0.5-25, 0.5-27.5, 0.5-30, 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-24.5, 27.5-32.5, 2-20, t 2.5-22.5, or 9.5-21.5 mg/m², of the body surface area. In some embodiments, plinabulin is administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, plinabulin is administered at a dose less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, or 40 mg/m² of the body surface area. In some embodiments, plinabulin is administered at a dose greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, or 50 mg/m² of the body surface area. In some embodiments, plinabulin is administered at a dose of about 10, 13.5, 20, or 30 mg/m² of the body surface area. In some embodiments, plinabulin is administered at a dose of about 20 mg/m² of the body surface area.

In some embodiments, the plinabulin dose is about 5 mg-100 mg, or about 10 mg-80 mg. In some embodiments, the plinabulin dose is about 15 mg-100 mg, or about 20 mg-80 mg. In some embodiments, plinabulin is administered at a dose in the range of about 15 mg-60 mg. In some embodiments, the plinabulin dose is about 0.5 mg-3 mg, 0.5 mg-2 mg, 0.75 mg-2 mg, 1 mg-10 mg, 1.5 mg-10 mg, 2 mg-10 mg, 3 mg-10 mg, 4 mg-10 mg. 1 mg-8 mg, 1.5 mg-8 mg, 2 mg-8 mg, 3 mg-8 mg, 4 mg-8 mg, 1 mg-6 mg, 1.5 mg-6 mg, 2 mg-6 mg, 3 mg-6 mg, or about 4 mg-6 mg. In some embodiments, plinabulin is administered at about 2 mg-6 mg or 2 mg-4.5 mg. In some embodiments, plinabulin is administered at about 5 mg-7.5 mg. 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg. 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg. 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg. 9 mg-54 mg, 9 mg-56 mg, 9 mg-58 mg, 9 mg-60 mg. 10 mg-12 mg, 10 mg-14 mg. 10 mg-15 mg. 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg. 10 mg-42 mg, 10 mg-44 mg, 10 mg-46 mg, 10 mg-48 mg, 10 mg-50 mg, 10 mg-52 mg. 10 mg-54 mg, 10 mg-56 mg, 10 mg-58 mg, 10 mg-60 mg, 12 mg-14 mg. 12 mg-15 mg. 12 mg-16 mg, 12 mg-18 mg, 12 mg-20 mg, 12 mg-22 mg, 12 mg-24 mg. 12 mg-26 mg, 12 mg-28 mg, 12 mg-30 mg. 12 mg-32 mg, 12 mg-34 mg. 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg. 12 mg-54 mg, 12 mg-56 mg, 12 mg-58 mg. 12 mg-60 mg. 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg. 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, mg-42 mg, 20 mg-44 mg, 20 mg-46 mg, 20 mg-48 mg, 20 mg-50 mg, 20 mg-52 mg, 20 mg-54 mg, mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, mg-38 mg. 30 mg-40 mg, 30 mg-42 mg, 30 mg-44 mg, 30 mg-46 mg, 30 mg-48 mg, 30 mg-50 mg, mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg. 50 mg-56 mg, 50 mg-58 mg, 50 mg-60 mg, 52 mg-54 mg, 52 mg-56 mg, 52 mg-58 mg, or 52 mg-60 mg. In some embodiments, plinabulin dose is greater than about 0.5 mg, 1 mg, 1.5 mg, 2 mg, 3 mg, 4 mg, 5 mg, 6 mg, 7 mg, 8 mg, 9 mg, about 10 mg, about 12.5 mg, about 13.5 mg, about 15 mg, about 17.5 mg, about 20 mg, about 22.5 mg, about 25 mg, about 27 mg, about 30 mg, or about 40 mg. In some embodiments, the plinabulin dose is about less than about 1 mg, 1.5 mg. 2 mg, 3 mg, 4 mg, 5 mg, 6 mg, 7 mg, 8 mg, 9 mg, about 10 mg, about 12.5 mg, about 13.5 mg, about 15 mg, about 17.5 mg, about 20 mg, about 22.5 mg, about 25 mg, about 27 mg, about 30 mg, about 40 mg, or about 50 mg.

In some embodiments, the treatment schedule includes administration of a plinabulin or a salt thereof concurrent with a blood transfusion. In some embodiments, the treatment schedule includes administration of a plinabulin or a salt thereof prior to a blood transfusion. In some embodiments, the treatment schedule includes administration of plinabulin or a salt thereof at least 1 minute prior to transfusion, at least 5 minutes prior to transfusion, at least 10 minutes prior to transfusion, at least 15 minutes prior to transfusion, at least 30 minutes prior to transfusion, at least 45 minutes prior to transfusion, at least 60 minutes prior to transfusion, at least 75 minutes prior to transfusion, at least 90 minutes prior to transfusion, at least 120 minutes prior to transfusion.

In some embodiments, the treatment schedule includes administration of a plinabulin or a salt thereof after a blood transfusion. In some embodiments, the treatment schedule includes administration of plinabulin or a salt thereof at least 1 minute after transfusion, at least 5 minutes after transfusion, at least 10 minutes after transfusion, at least 15 minutes after transfusion, at least 30 minutes after transfusion, at least 45 minutes after transfusion, at least 60 minutes after transfusion, at least 75 minutes after transfusion, at least 90 minutes after transfusion, at least 120 minutes after transfusion.

In some embodiments, the treatment schedule includes administration of a plinabulin or a salt thereof (e.g., the first, the second, the third, the fourth, the fifth, the sixth, the seventh, or the eighth) once every 3 weeks. In some embodiments, the treatment schedule includes administration of plinabulin or a salt thereof once every 1 week, 2 weeks, 3 weeks, 4 weeks, 5 weeks, 6 weeks, 7 weeks, or 8 weeks. In some embodiments, the treatment schedule includes administration of plinabulin or a salt thereof two times every 1 week, 2 weeks, 3 weeks, 4 weeks, 5 weeks, 6 weeks, 7 weeks, or 8 weeks. In some embodiments, the treatment schedule includes administration of plinabulin or a salt thereof once every 1 week in a treatment cycle of 1 week, 2 weeks, 3 weeks, 4 weeks, 5 weeks, 6 weeks, 7 weeks, or 8 weeks. In some embodiments, the treatment schedule includes administration of plinabulin or a salt thereof twice every 1 week in a treatment cycle of 1 week, 2 weeks, 3 weeks, 4 weeks, 5 weeks, 6 weeks, 7 weeks, or 8 weeks. In some embodiments, the treatment schedule includes administration plinabulin or a salt thereof three times (e.g., day 1, 2, 3, or day 1, 3, 5) every week in a treatment cycle of 1 week, 2 weeks, 3 weeks, 4 weeks, 5 weeks, 6 weeks, 7 weeks, or 8 weeks. In some embodiments, the treatment schedule includes administration of plinabulin or a salt thereof day 1, day 8, and day 15 of a 21-day treatment cycle. In some embodiments, the treatment schedule includes administration plinabulin or a plinabulin composition every day of the week for a week. In some embodiments, the treatment schedule includes administration plinabulin or a salt thereof every day of the week for 2 weeks, 3 weeks, or 4 weeks. In some embodiments, the treatment schedule includes administration plinabulin or a plinabulin composition on day 1 in weekly treatment. In some embodiments, the treatment schedule includes administration plinabulin or a salt thereof on day 1 and day 2 in weekly treatment. In some embodiments the treatment schedule includes administration plinabulin or a plinabulin composition on day 1, day 2, and day 3 in weekly treatment. In some embodiments, the treatment schedule includes administration plinabulin or a salt thereof on day 1, day 3, day 5 in weekly treatment. In some embodiments, the treatment schedule includes administration plinabulin or a salt thereof on day 1, day 2, day 3, and day 4 in weekly treatment. In some embodiments, the treatment schedule includes administration plinabulin or a salt thereof on day 1, day 2, day 3, day 4, and day 5 in weekly treatment. The treatment schedule includes administration plinabulin or a salt thereof on day 1, day 2, day 3, day 4, day 5, and day 6 in weekly treatment.

In some embodiments, the treatment schedule includes administration of a pharmaceutical composition comprising plinabulin once every 3 weeks. In some embodiments, the treatment schedule includes administration of plinabulin once every 1 week. 2 weeks, 3 weeks, 4 weeks, 5 weeks, 6 weeks, 7 weeks, or 8 weeks. In some embodiments, the treatment schedule includes administration of plinabulin two times every 1 week, 2 weeks, 3 weeks, 4 weeks, 5 weeks, 6 weeks, 7 weeks, or 8 weeks. In some embodiments, the treatment schedule includes administration of plinabulin once every 1 week in a treatment cycle of 1 week, 2 weeks, 3 weeks, 4 weeks, 5 weeks, 6 weeks, 7 weeks, or 8 weeks. In some embodiments, the treatment schedule includes administration of plinabulin twice every 1 week in a treatment cycle of 1 week, 2 weeks, 3 weeks, 4 weeks, 5 weeks, 6 weeks, 7 weeks, or 8 weeks. In some embodiments, the treatment schedule includes administration of plinabulin three times (e.g., day 1, 2, 3, or day 1, 3, 5) every 1 week in a treatment cycle of 1 week, 2 weeks, 3 weeks, 4 weeks, 5 weeks, 6 weeks, 7 weeks, or 8 weeks. In some embodiments, the treatment schedule includes administration of plinabulin day 1, day 8, and day 15 of a 21-day treatment cycle. In some embodiments, the treatment schedule includes administration of plinabulin every day of the week for a week. In some embodiments, the treatment schedule includes administration of plinabulin every day of the week for 2 weeks, 3 weeks, or 4 weeks. In some embodiments, the treatment schedule includes administration of plinabulin on day 1 in weekly treatment. In some embodiments, the treatment schedule includes administration of plinabulin on day 1 and day 2 in weekly treatment. In some embodiments the treatment schedule includes administration of plinabulin on day 1, day 2, and day 3 in weekly treatment. In some embodiments, the treatment schedule includes administration of plinabulin on day 1, day 3, day 5 in weekly treatment. In some embodiments, the treatment schedule includes administration of plinabulin on day 1, day 2, day 3, and day 4 in weekly treatment. In some embodiments, the treatment schedule includes administration of plinabulin on day 1, day 2, day 3, day 4, and day 5 in weekly treatment. the treatment schedule includes administration of plinabulin on day 1, day 2, day 3, day 4, day 5, and day 6 in weekly treatment.

### Combination Therapies

As disclosed elsewhere herein, some embodiments provide for coadministering a composition, such as plinabulin, described herein with an additional therapeutic agent. Combination therapies can include fixed combinations, in which two or more pharmaceutically active agents are in the same formulation; kits, in which two or more pharmaceutically active agents in separate formulations are sold in the same package, e.g., with instructions for co-administration; and free combinations in which the pharmaceutically active agents are packaged separately, but instruction for simultaneous or sequential administration are provided. Other kit components can include diagnostics, assays, multiple dosage forms for sequential or simultaneous administration, instructions and materials for reconstituting a lyophilized or concentrated form of the pharmaceutical composition, apparatus for administering the pharmaceutically active agents, and the like. For example, a pharmaceutical package is provided comprising a first drug substance which is a compound of embodiments disclosed elsewhere herein and at least one second drug substance, along with instructions for combined administration. A pharmaceutical package is also provided comprising a compound of embodiments disclosed elsewhere herein along with instructions for combined administration with at least one second drug substance. Also provided is a pharmaceutical package comprising at least one second drug substance along with instructions for combined administration with a compound of the present disclosure.

Treatment with combinations according to embodiments disclosed elsewhere herein may provide improvements or superior outcome compared with treatments by either component of the combination alone. For example, a pharmaceutical combination comprising an amount of a compound of some embodiments and an amount of a second drug substance can be employed, wherein the amounts are appropriate to produce a synergistic therapeutic effect. In some embodiments, a pharmaceutical combination comprising a plinabulin and a second therapeutic agent produce a synergistic therapeutic effect. In some embodiments, the second therapeutic agent is vitamin or mineral. In some embodiments, the second therapeutic agent is soluble iron.

In some aspects, the method of treating an iron disorder further comprises administering a metal chelator. In some embodiments, a metal chelator is administered with plinabulin. In some embodiments, a metal chelator is administered before plinabulin. In some embodiments, a metal chelator is administered after plinabulin. In some embodiments, the metal chelator is an iron chelator. In some embodiments, the iron chelator is deferasirox, dimercaptosuccinic acid, deferoxamine, deferiprone, or trientine.

In some embodiments, the compositions described herein can be administered or used in combination with other treatments such as radiation or biologic therapies. In some embodiments, the radiation is administered before plinabulin. In some embodiments, the radiation is administered after plinabulin. In some embodiments, the biologic therapies are administered before plinabulin. In some embodiments, the biologic therapies are administered after plinabulin. In some embodiments, the method of treating, reducing, or ameliorating an iron disorder further includes administering a monoclonal antibody. In some embodiments, the monoclonal antibody specifically recognizes an ERFE polypeptide. In some embodiments, the monoclonal antibody is ferroportin. In some embodiments, the method of treating, reducing, or ameliorating an iron disorder further includes administering hepcidin or a hepcidin analog.

In some embodiments, the method of treating, reducing, or ameliorating an iron disorder further includes administering a phlebotomy. In some embodiments, the method of treating, reducing, or ameliorating an iron disorder further includes administering a venesection.

In some embodiments, the method of treating, reducing, or ameliorating an iron disorder further includes a dietary restriction. In some embodiments, the dietary restriction may include avoiding supplements that contain iron, avoiding supplements that contain vitamin C, reducing iron-rich and iron-fortified foods, avoiding uncooked fish and shellfish, limiting alcohol intake, or a combination thereof.

### Pharmaceutical Compositions

Some embodiments include pharmaceutical compositions comprising plinabulin or a pharmaceutically effective salt thereof.

In some embodiments, the pharmaceutical compositions described above comprise an additional agent that acts to reduce an iron disorder. In some embodiments, the additional agent is an iron chelator. For example, in some embodiments, the pharmaceutical compositions include deferasirox, dimercaptosuccinic acid, deferoxamine, deferiprone, or trientine.

Other embodiments include two or more separate pharmaceutical compositions, one of which comprises plinabulin or a pharmaceutically acceptable salt thereof, and one or more other pharmaceutical compositions that comprise additional therapeutic agents.

In some embodiments, the compositions described above can further include one or more pharmaceutically acceptable diluents. In some embodiments, the pharmaceutically acceptable diluent can include Kolliphor® (Polyethylene glycol (15)-hydroxystearate). In some embodiments, the pharmaceutically acceptable diluent can include propylene glycol. In some embodiments, the pharmaceutically acceptable diluents can include Kolliphor® and propylene glycol. In some embodiments, the pharmaceutically acceptable diluents can include Kolliphor® and propylene glycol, wherein the Kolliphor® is about 40% by weight and propylene glycol is about 60% by weight based on the total weight of the diluents. In some embodiments, the composition can further include one or more other pharmaceutically acceptable excipients.

In some embodiments, the compositions described above can further include one or more pharmaceutically acceptable carrier or excipient. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerin, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The compositions described herein are preferably provided in unit dosage form. As used herein, a “unit dosage form” is a composition containing an amount of a compound or composition that is suitable for administration to an animal, preferably a mammalian subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, although a single administration is not specifically excluded. The skilled artisan will recognize that the formulation does not specifically contemplate the entire course of therapy and such decisions are left for those skilled in the art of treatment rather than formulation.

Standard pharmaceutical formulation techniques can be used to make the pharmaceutical compositions described herein, such as those disclosed in Remington's The Science and Practice of Pharmacy, 21st Ed., Lippincott Williams & Wilkins (2005), incorporated herein by reference in its entirety.

The compositions as described above may be in any of a variety of suitable forms for a variety of routes for administration, for example, for oral, sublingual, buccal, nasal, rectal, topical (including transdermal and intradermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the activity of the compound or composition. The amount of carrier employed in conjunction with the compound or composition is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules (e.g., liquid gel capsule and solid gel capsule), granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject composition is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium (EDTA), although other chelating agents may also be used in place or in conjunction with it.

For topical use, creams, ointments, gels, solutions or suspensions, etc., containing the composition disclosed herein are employed. Topical formulations may generally be comprised of a pharmaceutical carrier, co-solvent, emulsifier, penetration enhancer, preservative system, and emollient.

For intravenous administration, the compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one or more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan. In some embodiments, a single dose of Plinabulin or other therapeutic agent may be from about 5 mg/m² to about 150 mg/m² of body surface area, from about 5 mg/m² to about 100 mg/m² of body surface area, from about 10 mg/m² to about 100 mg/m² of body surface area, from about 10 mg/m² to about 80 mg/m² of body surface area, from about 10 mg/m² to about 50 mg/m² of body surface area, from about 10 mg/m² to about 40 mg/m² of body surface area, from about 10 mg/m² to about 30 mg/m² of body surface area, from about 13.5 mg/m² to about 100 mg/m² of body surface area, from about 13.5 mg/m² to about 80 mg/m² of body surface area, from about 13.5 mg/m² to about 50 mg/m² of body surface area, from about 13.5 mg/m² to about 40 mg/m² of body surface area, from about 13.5 mg/m² to about 30 mg/m² of body surface area, from about 15 mg/m² to about 80 mg/m² of body surface area, from about 15 mg/m² to about 50 mg/m² of body surface area, or from about 15 mg/m² to about 30 mg/m² of body surface area. In some embodiments, a single dose of Plinabulin or other therapeutic agent may be from about 13.5 mg/m² to about 30 mg/m² of body surface area. In some embodiments, a single dose of Plinabulin or other therapeutic agent may be about 5 mg/m², about 10 mg/m², about 12.5 mg/m², about 13.5 mg/m², about 15 mg/m², about 17.5 mg/m², about 20 mg/m², about 22.5 mg/m², about 25 mg/m², about 27.5 mg/m², about 30 mg/m², about 40 mg/m², about 50 mg/m², about 60 mg/m², about 70 mg/m², about 80 mg/m², about 90 mg/m², or about 100 mg/m², of body surface area.

In some embodiments, a single dose of Plinabulin or other therapeutic agent may be from about 5 mg to about 300 mg, from about 5 mg to about 200 mg, from about 7.5 mg to about 200 mg, from about 10 mg to about 100 mg, from about 15 mg to about 100 mg, from about 20 mg to about 100 mg, from about 30 mg to about 100 mg, from about 40 mg to about 100 mg, from about 10 mg to about 80 mg, from about 15 mg to about 80 mg, from about 20 mg to about 80 mg, from about 30 mg to about 80 mg, from about 40 mg to about 80 mg, from about 10 mg to about 60 mg, from about 15 mg to about 60 mg, from about 20 mg to about 60 mg, from about 30 mg to about 60 mg, or from about 40 mg to about 60 mg. In some embodiments, a single dose of Plinabulin or other therapeutic agent may be from about 20 mg to about 60 mg, from about 27 mg to about 60 mg, from about 20 mg to about 45 mg, or from about 27 mg to about 45 mg. In some embodiments, a single dose of Plinabulin or other therapeutic agent may be about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Example

Method. Non-metastatic Breast Cancer patients receiving TAC (taxotere, doxorubicin, cyclophosphamide) also received plinabulin at a dose of 10 (n=15), 20 (n=15) or 30 (n=12) mg/m². Hematology safety assessments were done at predose, and post-dose plinabulin at Day 2, 3, 6, 7, 8, 9, 10, 11, 12, 13 and 15, as predefined prospectively per protocol. Plinabulin was given on Day 1 as a single dose per 21-day cycle. In analyzing the hematology assessments, it was surprisingly discovered the plinabulin increased haptoglobin levels.

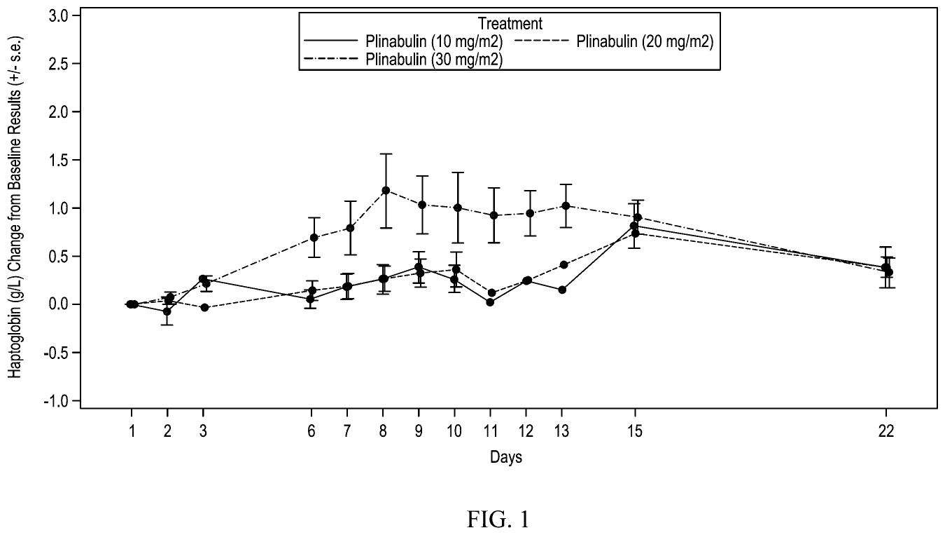

Results. The 30 mg/m² Plinabulin dose was the most effective dose for increasing plasma haptoglobin levels vs the lower doses (p<0.01); baseline mean (SD) haptoglobin was 1.32 (0.453) g/L and after Plinabulin 30 mg/m² dosing, maximum haptoglobin levels increased with more than 2-fold with a mean (SD) increase of 1.61 (1.29) g/L (p<0.0005). Hp increase occurred within 2 days of plinabulin dosing (P<0.03) and lasted for >3 weeks (FIG. 1)

Based on this data, a single dose of plinabulin 30 mg/m² produces a rapid (within 2 days) and sustained (for >3 weeks) increase in haptoglobin levels that is statistically and clinically significant. Since haptoglobin neutralizes free hemoglobin, plinabulin's ability to increase/double haptoglobin levels can be used for the prevention of hemolysis dependent iron overload.

What is claimed is:

1. A method of preventing an iron disorder in a mammal, wherein the method comprises administering to the mammal in need thereof a therapeutically effective amount of plinabulin.

2. The method of claim 1, wherein the iron disorder is associated with chronic blood transfusions.

3. The method of claim 1, wherein the iron disorder is associated with disease.

4. The method of claim 2, wherein the disease is selected from the group consisting of neurodegenerative diseases, cardiovascular diseases, inflammatory diseases, cancer, insulin resistance, non-alcoholic liver disease, alcoholic liver disease, and an infectious disease.

5. The method of claim 3, wherein the neurodegenerative disease is ALS, a prion disease, Parkinson's disease, or Alzheimer's disease.

6. The method of claim 3, wherein the cardiovascular disease is atherosclerosis, ishemic cerebrovascular disease, or ischemic stroke.

7. The method of claim 3, wherein the cancer is head and neck cancer, lung cancer, stomach cancer, colon cancer, pancreatic cancer, prostate cancer, breast cancer, kidney cancer, bladder cancer, ovary cancer, cervical cancer, melanoma, glioblastoma, myeloma, lymphoma, leukemia, renal cell carcinoma, malignant melanoma, non-small cell lung cancer (NSCLC), ovarian cancer, Hodgkin's lymphoma or squamous cell carcinoma.

8. The method of any one of claims 1 to 7, wherein iron disorder is selected from iron overload disorder or an anemic condition.

9. The method of claim 8, wherein the iron overload is associated with oxyradical formation.

10. The method of claim 9, wherein the oxyradical formation is associated with an ischemia-reperfusion.

11. The method of claim 10, wherein ischemia-reperfusion occurs during an organ transplant, cardiac PCI procedure, or a reperfusion procedure.

12. The method of claim 8, wherein the iron overload disorder is a primary iron overload disorder.

13. The method of claim 12, wherein the primary iron overload disorder is selected from the group consisting of hereditary hemochromatosis, juvenile hemochromatosis, ferroportin disease, neonatal hemochromatosis, Bantu siderosis, African iron overload, gracile syndrome, ataxia, and Friedreich Ataxia.

14. The method of any one of claims 8-13, wherein the iron overload disorder is a secondary iron overload disorder.

15. The method of claim 14, wherein the secondary iron overload disorder is selected from thalassemia, hypochromic microcytic anemia, sickle cell anemia, microcytic iron loading anemia, hereditary sideroblastic anemia, congenital dyserythropoeitic anemia, Porphyria cutanea tarda, pyruvate kinase deficiency, hereditary atransferrinemia, ceruloplasmin deficiency, myelodysplastic syndromes, pulmonary hemosiderosis, aceruloplasminemia and x-linked sideroblastic anemia.

16. The method of claim 8, wherein the anemic condition is selected from the group consisting of hemolytic anemia, thalassemia, hereditary spherocytosis, hereditary elliptocytosis, glucose-6-phosphate dehydrogenase deficiency, pyruvate kinase deficiency, immune hemolytic anemia, alloimmune hemolytic anemia, drug-induced hemolytic anemia, mechanical hemolytic anemia, paroxysmal nocturnal hemoglobinuria, anemia of chronic disease, anemia, aplastic anemia, congenital hypoplastic anemia, Diamond-Blackfan anemia, Fanconi anemia, iron deficiency anemia, anemia of abnormal RBC size, megaloblastic anemia, microcytic anemia, vitamin deficiency anemia, pernicious anemia, anemia of RBC mutation, sideroblastic anemia, and sickle cell anemia.

17. The method of any one of claims 2 to 16, wherein the iron overload is an accumulation of iron in a body tissue.

18. The method of claim 17, wherein the body tissue is selected from myocardium, liver, and endocrine organs.

19. The method of any one of claims 1-18, further comprising administering a metal chelator.

20. The method of claim 19, wherein the metal chelator is an iron chelator.

21. The method of claim 20, wherein the iron chelator is selected from is deferasirox, dimercaptosuccinic acid, deferoxamine, deferiprone, or trientine.

22. The method of any one of claims 1-21, wherein prior to treatment, the mammal exhibits an elevated haptoglobin level.

23. The method of any one of claims 1-22, wherein the prior to treatment, the mammal exhibits a decreased hemoglobin level.

24. The method of any one of claims 1-23, further comprising identifying the mammal as having the iron disorder prior to administration of plinabulin.

25. A method of increasing haptoglobin in a mammal, wherein the method comprises administering to the mammal in need thereof a therapeutically effective amount of plinabulin.

26. The method of claim 25, further comprising identifying the mammal as having a decreased haptoglobin level prior to administration of plinabulin.

27. A method of reducing iron overload, comprising administering plinabulin or a pharmaceutically acceptable salt thereof to a subject in need thereof.

28. The method of claim 27, further comprising identifying the subject as having iron overload prior to administration of plinabulin.

29. A method of decreasing free plasma hemoglobin in a subject, comprising administering plinabulin or a pharmaceutically acceptable salt thereof to a subject in need thereof.

30. The method of claim 29, further comprising identifying the subject as having an elevated free plasma hemoglobin level prior to administration of plinabulin.

31. A method of treating an iron disorder, comprising administering plinabulin or a pharmaceutically acceptable salt thereof to a subject in need thereof.

32. The method of claim 31, further comprising identifying the subject as at risk for developing the iron disorder prior to administration of plinabulin.

33. The method of any one of claims 1-31, wherein administration of plinabulin reduces an incidence of the iron disorder by at least 10%.

34. The method of any one of claims 1-32, wherein administration of plinabulin reduces an incidence of the iron disorder by at least 30%.

35. The method of any one of claims 1-34, wherein administration of plinabulin reduces a duration of the iron disorder by at least 2 times.

36. The method of claim 1-34, wherein administration of plinabulin reduces a duration of the iron disorder by at least 30%.

37. The method of any one of claims 1-36, wherein the plinabulin is administered at a dose in the range of about 2.5 mg/m² to about 40 mg/m².

38. The method of any one of claims 1-36, wherein the plinabulin is administered at a dose in the range of about 8.5 mg/m² to about 35 mg/m².

39. The method of any one of claims 1-36, wherein the plinabulin is administered at a dose in the range of about 20 mg/m² to about 35 mg/m².

40. The method of any one of claims 1-36, wherein the plinabulin is administered at a dose in the range of about 25 mg/m² to about 35 mg/m².

41. The method of any one of claims 1-36, wherein the plinabulin is administered at a dose about 30 mg/m².

42. The method of any one of claims 1-41, wherein the plinabulin is administered simultaneously with a blood transfusion.

43. The method of any one of claims 1-41, wherein the plinabulin is administered prior to a blood transfusion.

44. The method of any one claims 1-41, wherein the plinabulin is administered after a blood transfusion.

45. A method of transfusing red blood cells, the method comprising:
transfusing red blood cells into a subject, wherein the red blood cells have been stored for longer than 30 days; and
administering plinabulin to the subject, wherein the plinabulin is administered prior to, during, or after the transfusion.

46. A method of treating or reducing an iron disorder in a subject receiving a cancer drug or cancer therapy, the method comprising: administering an effective amount of plinabulin.

47. The method of claim 46, wherein the cancer drug or cancer therapy is selected from docetaxel, paclitaxel, TC or TAC.

48. The method of claim 46 or 47, wherein the amount of plinabulin administered is a dose from about 10 mg/m² to about 50 mg/m².

49. The method of any one of claims 46 to 48, wherein the plinabulin is administered on day 1 of a 21 day cancer drug or cancer therapy cycle.

50. The method of any one of claims 46 to 49, wherein the subject's haptoglobulin levels are increased after administration of plinabulin.
